(19) United States
(12) Patent Application Publication
(10) Pub. No.: US 2009/0198101 A1
(43) Pub. Date: Aug. 6, 2009

(54) CAPSULE ENDOSCOPE

(75) Inventors: Hidetake SEGAWA, Tokyo (JP); Satomi KOBAYASHI, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/367,728

(22) Filed: Feb. 9, 2009

Related U.S. Application Data
(63) Continuation of application No. PCT/JP2007/064668, filed on Jul. 26, 2007.

(30) Foreign Application Priority Data
Aug. 9, 2006 (JP) 2006-217205
Oct. 30, 2006 (JP) 2006-293947

(51) Int. Cl. A61B 1/04

(52) U.S. Cl. 600/109

(57) ABSTRACT

A capsule endoscope includes a capsule body to be introduced into a subject; and a center-of-gravity setting unit for setting a center of gravity so as to maintain a posture of the capsule body relative to a direction of gravity constant. The capsule endoscope also includes an imaging unit arranged in the capsule body such that an imaging field having an optical axis in an oblique direction pointing upward from a horizontal direction is formed with a posture of the capsule body maintained constant relative to the direction of gravity, for imaging a body cavity image.

### CROSS-REFERENCE TO RELATED APPLICATIONS

This application is a continuation of PCT international application Ser. No. PCT/JP2007/064668 filed on Jul. 26, 2007 which designates the United States, incorporated herein by reference, and which claims the benefit of priority from Japanese Patent Application No. 2006-217205, filed on Aug. 9, 2006, and Japanese Patent Application No. 2006-293947, filed on Oct. 30, 2006, both incorporated herein by reference.

### BACKGROUND OF THE INVENTION

1. Field of the Invention

The present invention relates to, for example, a capsule endoscope introduced into a lumen organ such as a large intestine in a subject for obtaining an intra-subject image.

2. Description of the Related Art

Recently, in a field of the endoscope, the capsule endoscope equipped with an imaging function and a radio communication function has appeared. The capsule endoscope has a configuration to move in organs (in a body cavity) such as an esophagus, a stomach, and a small intestine, with a peristaltic action thereof for observation (examination) during an observation period after being swallowed from a mouth of the subject (human body), until naturally exiting the subject, and to sequentially image using the imaging function (see Japanese Patent Application Laid-Open No. 2006-20702, for example).

### SUMMARY OF THE INVENTION

A capsule endoscope according to an aspect of the present invention includes a capsule body to be introduced into a subject; a center-of-gravity setting unit for setting a center of gravity so as to maintain a posture of the capsule body relative to a direction of gravity constant; and an imaging unit arranged in the capsule body such that an imaging field having an optical axis in an oblique direction pointing upward from a horizontal direction is formed with a posture of the capsule body maintained constant relative to the direction of gravity, for imaging a body cavity image.

A capsule endoscope according to another aspect of the present invention includes a capsule body having a substantially cylindrical shape to be introduced into a subject; a center-of-gravity setting unit for setting a center of gravity such that the capsule body maintains a posture relative to a direction of gravity constant without rotating in a circumferential direction, on a position eccentric from a longitudinal axis of the capsule body; and an imaging unit arranged in the capsule body such that an imaging field having an optical axis in an oblique direction pointing upward from a horizontal direction is formed with a posture of the capsule body maintained constant relative to the direction of gravity, for imaging a body cavity image.

A capsule endoscope according to still another aspect of the present invention includes a capsule body of which specific gravity including a built-in component is set so as to sink in liquid introduced into a subject; a center-of-gravity setting unit for setting a center of gravity so as to maintain a posture of the capsule body relative to a direction of gravity constant in a state in which the capsule body sinks in the liquid introduced into the subject; and an imaging unit arranged in the capsule body such that an imaging field having an optical axis in an oblique direction pointing upward from a horizontal direction is formed with a posture of the capsule body maintained constant relative to the direction of gravity, for imaging a body cavity image.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic configuration diagram showing a capsule endoscope according to a first embodiment of the present invention;

FIG. 2-1 is a schematic diagram showing a modification using a battery as a center-of-gravity setting unit;

FIG. 2-2 is a schematic configuration diagram showing a modification using a necessary built-in component as the center-of-gravity setting unit;

FIG. 3 is a schematic configuration diagram showing a binocular type configuration example of the capsule endoscope shown in FIG. 1;

FIG. 4 is a schematic configuration diagram showing the capsule endoscope according to a second embodiment of the present invention;

FIG. 5 is a schematic configuration diagram showing the capsule endoscope according to a third embodiment of the present invention;

FIG. 6 is a schematic configuration diagram showing the capsule endoscope according to a modification;

FIG. 7 is an enlarged schematic configuration diagram showing a vicinity of an end on an imaging side of the capsule endoscope according to a fourth embodiment of the present invention;

FIG. 8 is an illustrative diagram showing a diffusion function of illumination light by a diffuser plate formed of a transmissive member;

FIG. 9 is a characteristic diagram showing a light intensity distribution characteristic on a liquid surface of an LED as a light source of the fourth embodiment and a transmissivity distribution characteristic of a diffuser plate;

FIG. 10-1 is an illustrative diagram showing an example of a configuration of the diffuser plate having a predetermined transmissivity distribution characteristic;

FIG. 10-2 is an illustrative diagram showing another example of the configuration of the diffuser plate having a predetermined transmissivity distribution characteristic;

FIG. 10-3 is an illustrative diagram showing yet another example of the configuration of the diffuser plate having a predetermined transmissivity distribution characteristic; and

FIG. 11 is a schematic configuration diagram showing a modification using the diffuser plate formed of a reflective member.

### DETAILED DESCRIPTION OF THE PREFERRED EMBODIMENTS

Hereinafter, embodiments of a capsule endoscope according to the present invention will be described in detail with reference to the drawings. Meanwhile, the present invention is not limited to the embodiments, and various modifications may be made without departing from the spirit of the invention.

### First Embodiment

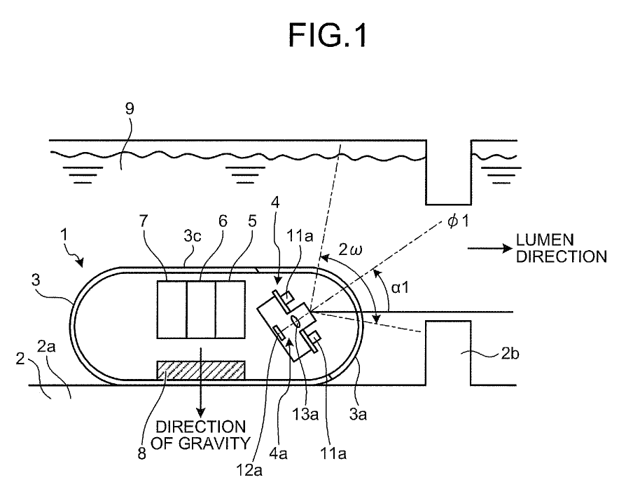

FIG. 1 is a schematic configuration diagram showing the capsule endoscope according to a first embodiment of the present invention. A capsule endoscope 1 of the first embodiment is preferable for imaging and observing inside a body cavity of a subject 2, especially, a large lumen organ such as a large intestine 2a, and is provided with a capsule body 3 insertable into the body cavity of the subject 2, and a built-in components such as an imaging unit 4, a radio transmitter 5, a battery 6, an image processor 7, and a spindle 8, built in the capsule body 3.

The capsule body 3 has a size swallowable from an oral cavity of the subject 2 to the body cavity, and forms an external case in a substantially cylindrical shape for liquid-tightly sealing an interior portion thereof by elastically fitting an end cover 3a in a substantially spherical shape and having transparency or translucency, and a body section cover 3c in a cylindrical shape formed of a colored material through which visible light cannot pass.

Here, the capsule endoscope 1 of the first embodiment moves while sinking in liquid 9 introduced into the subject 2, and specific gravity of the capsule body 3 including the built-in components 4 to 8 thereof is set larger than that of the liquid 9. The liquid 9 is swallowable from the oral cavity of the subject 2, and is transparent relative to a wavelength of a light source used for imaging in the imaging unit 4, and potable water of which specific gravity is near 1 is used, as an example, in the first embodiment. Therefore, the specific gravity of the capsule endoscope 1 is equal to or larger than 1.

The spindle 8 is not a specially required member for a function of the capsule endoscope 1, unlike required members for the function of the capsule endoscope 1 such as the imaging unit 4, the radio transmitter 5, the battery 6, and the image processor 7; however, this is provided for adjusting the specific gravity, and is provided as a center-of-gravity setting unit for setting a center of gravity to a position eccentric from a longitudinal axis so as to maintain a posture of the capsule body 3 sinking in the liquid 9 constant. In the first embodiment, by arranging the spindle 8 on an eccentric position on a lateral axis (direction across a central portion of the longitudinal direction) of the capsule body 3 and on an inner wall surface of the capsule body 3, the center of gravity also is set on the position on the lateral axis and eccentric toward the spindle 8, and the capsule body 3 is set to maintain the posture sinking in the liquid 9 always in a horizontal state without rotating in a circumferential direction in a horizontal portion of the large intestine 2a. Thereby, the posture of the capsule body 3 relative to the direction of gravity is maintained constant such that a side on which the spindle 8 is arranged is a bottom surface portion, and the bottom surface portion is always located on a lower side in the direction of gravity indicated by an arrow, as the posture sinking in the liquid 9.

Also, the imaging unit 4 is imaging means for imaging a body cavity image, and includes an imaging optical system 4a arranged on an end cover 3a side. The imaging optical system 4a is provided with a plurality of light sources 11a each formed of an LED or the like to illuminate an imaging site, an imaging device 12a such as CCD and CMOS imagers for receiving reflected light from the imaging site by illumination light of the light sources 11a to image the body cavity image, and an image forming lens 13a for forming an optical image of the imaging site on the imaging device 12a. A maximum imaging angle of the imaging optical system 4a is set to 2ω.

Also, in the first embodiment, the imaging optical system 4a is configured as a diagonal imaging optical system in which an optical axis φ1 is set in an oblique direction pointing upward inclined by a diagonal angle α1 from a horizontal direction (longitudinal axis direction of the capsule body 3) in the capsule body 3 of which posture sinking in the liquid 9 is always maintained in the horizontal state and a vertical direction thereof is specified in the horizontal portion of the large intestine 2a. Thereby, an imaging field of the imaging optical system 4a also is set to be in the oblique direction pointing upward, which is a direction opposite to the direction of gravity. As shown in FIG. 1, it is set that the imaging field is generally formed in a lumen direction, as seen from the capsule body 3 on a position having sunk to the inner wall of the large intestine 2a, which is the larger lumen, in the liquid 9.

Here, in the first embodiment, it is set to satisfy a condition, α1<2ω/2 where the diagonal angle of the optical axis φ1 of the imaging optical system 4a is α1 and the maximum angle of view is 2ω. By satisfying this condition, the optical axis φ1 of the imaging optical system 4a does not point too much upward, and an area in a direction downward from the horizontal direction also is included in the imaging field. This allows an observation over an entire lumen including the inner wall bottom surface side portion of the large intestine 2a.

The image processor 7 is for applying a required image process to the body cavity image imaged by the imaging device 12a, and the radio transmitter 5 is for radio outputting data of the body cavity image imaged by the imaging device 12a and to which the required image process is applied by the image processor 7 to a receiver (not shown) arranged outside of the subject 2. The battery 6 is for supplying power required for an electric system driving unit such as the light sources 11a and the imaging device 12a built in the capsule body 3.

Next, the observation of the body cavity image using the capsule endoscope 1 of the first embodiment will be described. Basically, the liquid 9 and the capsule endoscope 1 are swallowed from the oral cavity to fill the large intestine 2a, which is a target site in the subject 2, with the liquid 9, and the body cavity image is imaged by the imaging optical system 4a while allowing the capsule endoscope 1 to move in the filled liquid 9 in the sinking state to observe.

Here, the capsule endoscope 1 is set so as to be eccentric toward the lower portion side in the lateral axis direction by the spindle 8, so that the capsule endoscope 1 is always maintained in the posture horizontal relative to the liquid 9 introduced into the horizontal portion of the target site, which is the large intestine 2a, as shown in FIG. 1, and this does not rotate except about the axis in the direction of gravity. That is to say, the capsule endoscope 1 is maintained in the state in which the bottom surface portion side having the spindle 8 is always located on the lower side in the direction of gravity and the vertical direction thereof is specified, and does not rotate in a circumferential direction. In such constant posture, the imaging field of the imaging optical system 4a is set in the lumen direction according to the optical axis φ1 set in the oblique direction pointing upward from the horizontal direction, so that it is possible to acquire the central portion of the large intestine 2a and to appropriately image and observe inside the large intestine 2a with reduced oversight. At that time, even when there is a projection 2b such as a fold, as shown in FIG. 1, the imaging and observing may be appropriately performed while illuminating a far side thereof with minimum light shielding by the projection 2b.

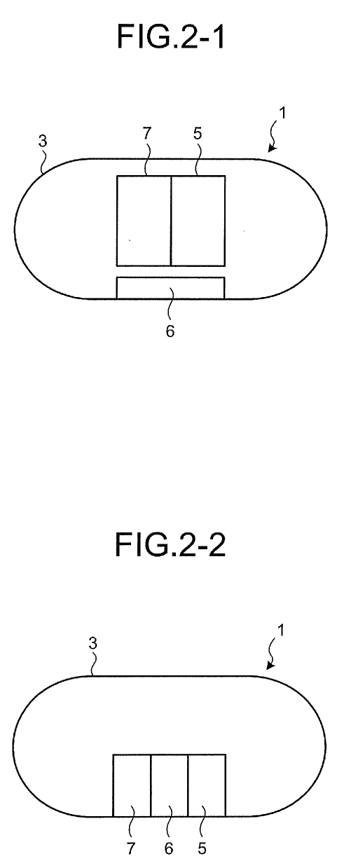

Meanwhile, in the first embodiment, although the center of gravity of the capsule endoscope 1 is set to the eccentric position by additionally providing the spindle 8, which is not necessary for the function of the capsule endoscope 1, as the center-of-gravity setting unit, it is also possible that the battery 6, which is the member necessary for the function of the capsule endoscope 1 and relatively heavy, is used as the center-of-gravity setting unit as shown in FIG. 2-1, for example, in place of the spindle 8, and is arranged on the position on the lateral axis and eccentric from the longitudinal axis of the capsule body 3, thereby eccentrically setting the center of gravity. Alternatively, as shown in FIG. 2-2, the center of gravity may be set on the position on the lateral axis and eccentric from the longitudinal axis of the capsule body 3, by combining the built-in components such as the radio transmitter 5, the battery 6, and the image processor 7, which are a plurality of members necessary for the function of the capsule endoscope 1 and arranging them on one side relative to the longitudinal axis. Such setting of the center of gravity is similar in the following embodiments.

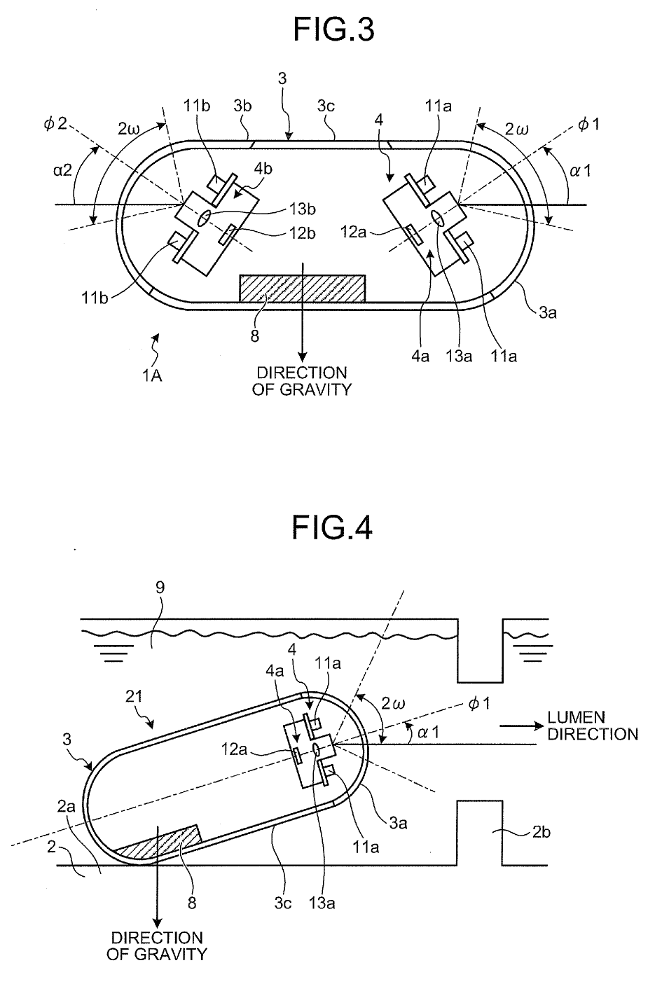

In addition, although it is described as an application example to a monocular capsule endoscope 1 provided with only the diagonal imaging optical system 4a in the first embodiment, it is also possible to configure as a binocular capsule endoscope 1A provided with an imaging optical system 4b similar to the imaging optical system 4a on the other end side in the longitudinal axis direction as shown in FIG. 3, and observe the body cavity image in front-back both directions to reduce the oversight. In FIG. 3, a reference numeral 3b indicates an end cover corresponding to the end cover 3a. Also, the imaging optical system 4b is provided with a plurality of light sources 11b each formed of the LED or the like for illuminating the imaging site, an imaging device 12b such as the CCD and CMOS imagers for receiving the reflected light from the imaging site by the illumination light of the light sources 11b to image the body cavity image, and an image forming lens 13b for forming the optical image of the imaging site on the imaging device 12b. The maximum imaging angle of the imaging optical system 4b is set to 2ω.

Here, the imaging optical system 4b is composed as the diagonal imaging optical system in which an optical axis φ2 is set in the oblique direction pointing upward inclined by the diagonal angle α2 from the horizontal direction (longitudinal axis direction of the capsule body 3) in the capsule body 3 of which posture sinking in the liquid 9 in the horizontal portion of the large intestine 2a is always maintained in the horizontal state and the vertical direction thereof is specified. Also, this is set so as to satisfy the condition, α2<2ω/2 where the diagonal angle of the optical axis φ2 of the imaging optical system 4b is α2 and the maximum angle of view is 2ω. Meanwhile, the diagonal angles α1 and α2 of the optical axes φ1 and φ2 of the imaging optical systems 4a and 4b, respectively, may be the same angle or different angles.

### Second Embodiment

FIG. 4 is a schematic configuration diagram showing the capsule endoscope according to a second embodiment of the present invention. The portion identical to the portion shown in FIG. 1 is indicated by the same reference numeral. A capsule endoscope 21 of the second embodiment is provided with the capsule body 3 insertable into the body cavity of the subject 2, and the built-in components such as the imaging unit 4 to the spindle 8 built in the capsule body 3. Here, the capsule endoscope 21 of the second embodiment moves in the state sinking in the liquid 9 introduced into the large intestine 2a of the subject 2 as in the case of FIG. 1, and the specific gravity of the capsule body 3 including the built-in components 4 to 8 is set so as to be larger (equal to or larger than 1) as compared to the liquid 9.

In addition, although the imaging unit 4 is configured as the monocular type having the imaging optical system 4a on one side in the longitudinal axis direction of the capsule body 3, in the capsule endoscope 21 of the second embodiment, the optical axis φ1 of the imaging optical system 4a is set in the longitudinal axis direction of the capsule body 3. Further, in the second embodiment, the center of gravity is set by the spindle 8 to the position eccentric from the longitudinal and lateral axes of the capsule body 3 to a backward corner portion (R) side. Thereby, it is set that the posture relative to the direction of gravity is maintained constant in the oblique state such that the portion on which the spindle 8 is arranged becomes the bottom surface portion to contact an inner wall and the end cover 3a side on which the imaging unit 4 is arranged floats from the inner wall in the state sinking in the liquid 9 in the horizontal portion in the large intestine 2a.

The inclination of the capsule body 3 in this case is set such that the optical axis φ1 of the imaging optical system 4a is in the oblique direction pointing upward, which is inclined by the angle α1 from the horizontal direction, in the capsule body 3 of which posture sinking in the liquid 9 is always maintained in the inclined state. Thereby, the imaging field of the imaging optical system 4a also is set to the oblique direction pointing upward, which is opposite to the direction of gravity. As shown in FIG. 4, as seen from the capsule body 3 in the position sinking on the inner wall of the large intestine 2a, which is the large lumen, in the liquid 9, it is set that the imaging field is generally formed in the lumen direction.

Here, in the second embodiment, it is set to satisfy the condition, α1<2ω/2 where the inclination angle of the optical axis φ1 of the imaging optical system 4a relative to the horizontal direction is α1 and the maximum angle of view is 2ω. By satisfying this condition, the optical axis φ1 of the imaging optical system 4a does not point too much upward, so that the area in the direction downward from the horizontal direction also is included in the imaging field.

The capsule endoscope 21 according to the second embodiment also has the effect similar to that of the capsule endoscope 1 according to the first embodiment.

### Third Embodiment

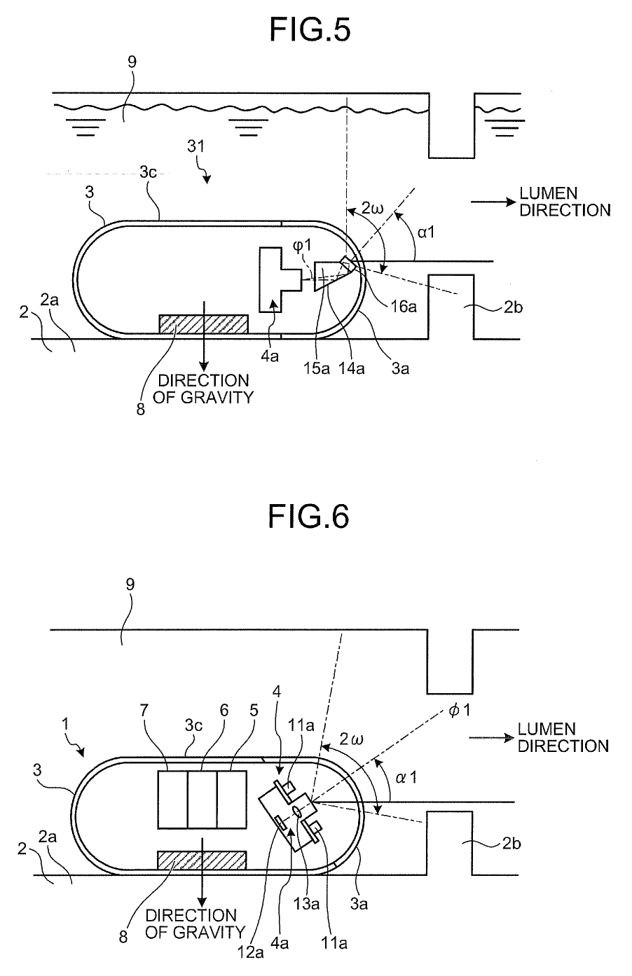

FIG. 5 is a schematic configuration diagram showing the capsule endoscope according to a third embodiment of the present invention. The portion identical to the portion shown in FIG. 1 is indicated by the same reference numeral. A capsule endoscope 31 according to the third embodiment is provided with the capsule body 3 insertable into the body cavity of the subject 2, and the built-in components such as the imaging unit 4 to the spindle 8 built in the capsule body 3. Here, the capsule endoscope 31 of the third embodiment moves in the state sinking in the liquid 9 introduced into the large intestine 2a of the subject 2, as in the case shown in FIG. 1, and the specific gravity of the capsule body 3 including the built-in components 4 to 8 is set so as to be larger (equal to or larger than 1) as compared to that of the liquid 9.

Although the imaging unit 4 is composed as the monocular type having the imaging optical system 4a on one side in the longitudinal axis direction of the capsule body 3, in the capsule endoscope 31 of the third embodiment, the optical axis φ1 of the imaging optical system 4a is set in the longitudinal axis direction of the capsule body 3, and a prism 15a is provided as a reflective member having a reflective surface 14a, which inclines the direction of imaging field of the imaging optical system 4a forward of an imaging direction of the imaging optical system 4a. Here, in the third embodiment, it is set to satisfy the condition, α1<2ω/2 where the inclination angle in the direction of imaging field of the imaging optical system 4a by the reflective surface 14a is α1 and the maximum angle of view is 2ω. By satisfying the condition, the direction of imaging field by the prism 15a does not point too much upward, so that the area in the downward direction from the horizontal direction also is included in the imaging field.

Here, in a case in which the reflective member is the prism 15a, it is required that a refractive index n of a prism glass material satisfies a total reflection condition on a prism refractive surface,

n≦1/sin(π/2−α1/2−ω)

in order to allow a light beam entering from the direction of field of view with the inclination angle α1 to enter the imaging device 12a without loss. It is desirable that the glass material of the prism 15a and the inclination angle of the refractive surface 14a are decided so as to satisfy the total reflection condition. Also, when realizing a wide angle of view, an appropriate glass material may not exist or a prism size may become larger, so that it is desirable to arrange a concave lens 16a on an incident surface of the prism 15a.

The capsule endoscope 31 according to the third embodiment also has the effect similar to that of the capsule endoscope 1 according to the first embodiment. Especially, according to the third embodiment, the optical axis φ1 of the imaging optical system 4a is set in the longitudinal axis direction of the capsule body 3, so that this can be easily realized only by appropriately setting the direction of the imaging field by adding the prism 15a or the like to the normal monocular capsule endoscope configuration.

Meanwhile, although the example of imaging and observing in the state that the capsule endoscopes 1, 21 and 31 are sunk in the liquid 9 introduced into the large intestine 2a has been described in the first to third embodiments, the large intestine 2a may be observed without using the liquid 9 also. FIG. 6 is a schematic configuration diagram showing a state in which the inside of the large intestine 2a is imaged and observed with the capsule endoscope 1 without using the liquid 9, as an example.

### Fourth Embodiment

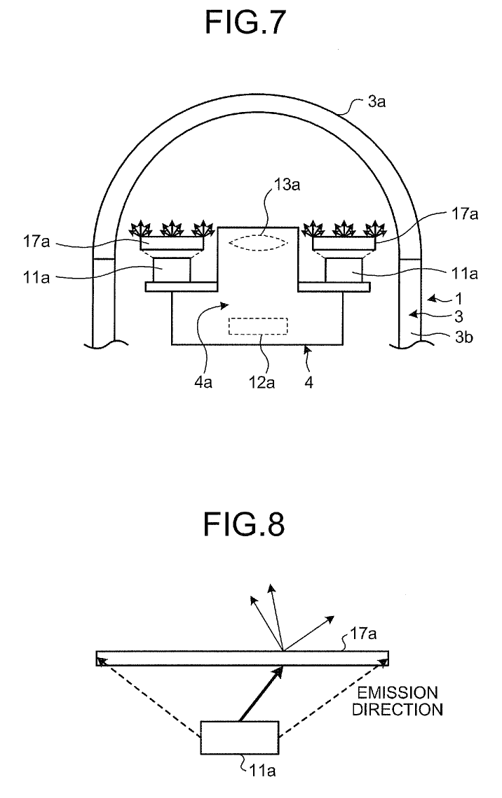

FIG. 7 is an enlarged schematic configuration diagram showing a vicinity of an end portion on an imaging side of the capsule endoscope according to a fourth embodiment of the present invention. The fourth embodiment shows, for example, a configuration example of the imaging optical system 4a according to the first embodiment. The imaging optical system 4a of the fourth embodiment is further provided with a diffuser plate 17a formed of a transmissive member as diffusing means arranged in an emission direction of the illumination light from each of the light sources 11a for diffusing the illumination light from the light sources 11a, as shown in FIG. 7. FIG. 8 is an illustrative view showing a diffusing function of the illumination light by the diffuser plate 17a formed of the transmissive member, and a diffusing agent, for example, of a granular substance may be added for improving diffuseness. Four light sources 11a using the LEDs are arranged around the image forming lens 13a so as to be in a cross shape relative to the center of the optical axis in the imaging unit 4, and the diffusing plates 17a are provided for each of the light sources 11a, and are arranged so as to cover an entire area of an imaging field E.

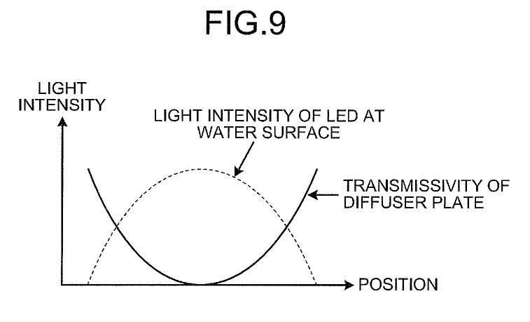

FIG. 9 is a characteristic diagram showing a light intensity distribution characteristic on the liquid 9 surface (water surface) and a transmissivity distribution characteristic of the diffuser plate 17a, when using the LEDs as the light sources 11a of the fourth embodiment. In the fourth embodiment, the diffuser plate 17a having the transmissivity distribution characteristic in which transmissivity increases from the central portion to the peripheral portion so as to be opposite to the light intensity characteristic of the LED (light source 11a) on the liquid 9 surface (water surface) of which light intensity decreases from the central portion to the peripheral portion is used. Such a diffuser plate 17a is arranged so as to be centered on the central portion of the emission direction of the illumination light from the LED.

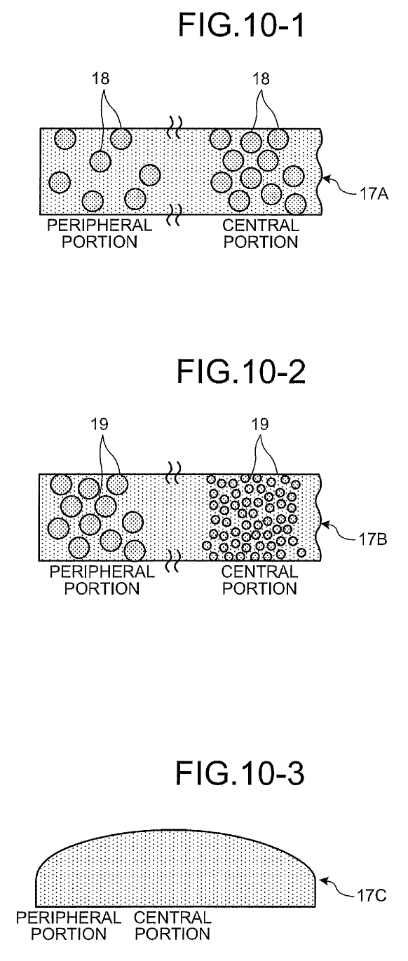

Here, a configuration example of the diffuser plate 17a having the transmissivity distribution characteristic as shown in FIG. 9 is illustrated in FIGS. 10-1 to 10-3. For example, as shown in FIG. 10-1, a diffuser plate 17A in which an additive amount of a diffusing agent 18 increases from the peripheral portion to the central portion may be used. Also, a diffuser plate 17B in which a size of the particle of the diffusing agent 19 to be added becomes smaller from the peripheral portion to the central portion, as shown in FIG. 10-2, may be used. Alternatively, a diffuser plate 17C of which thickness becomes larger from the peripheral portion to the central portion as shown in FIG. 10-3 may be used. In a case of the diffuser plate 17C, since this is not formed of a single crystal, this does not have the lens function even when this has the shape as shown.

In such a configuration, in the fourth embodiment, for example, a predetermined amount of liquid 9 is introduced into an observation target site such as the large intestine in the subject 2 to extend the same, and at the same time, the capsule endoscope 1 is inserted into the subject 2 and is allowed to float in the sinking state in the liquid 9 to extend the observation target site as shown in FIG. 1, and the inner wall or the like of the large intestine, which is the object, is illuminated by the illumination light from the light source 11a and is imaged by the imaging unit 4 to observe.

Here, in a case in which there is not the diffuser plate 17a in the imaging unit 4, the imaging field is directly illuminated by the LED (light source 11a) having the light intensity distribution characteristic in which the light intensity decreases from the central portion to the peripheral portion as shown in FIG. 9; however, in this case, the illumination light is reflected by the liquid surface of the liquid 9 as a boundary surface, and when imaging the object in gas, the illumination light brightly reflects as a light source image, thereby interfering with the observation. Especially, when the illumination light reflects on the boundary surface, the brightest central potion strongly reflects as a bright point, thereby interfering with the imaging of the imaging site.

Therefore, since the fourth embodiment has the diffuser plate 17a for diffusing the illumination light from the LED (light source 11a) in the emission direction of the illumination light from the LED (light source 11a), the illumination light from the LED (light source 11a) is diffused by the diffuser plate 17a to illuminate the imaging field, thereby preventing the light source image from being directly reflected by the boundary surface to reflect into the imaging device 12a when imaging the site in the subject in gas as the object from the liquid 9 across the boundary surface, so that the site of the subject may be appropriately observed. Especially, as in the fourth embodiment, to the normal LED (light source 11a) emitting the illumination light having the light intensity distribution characteristic in which the light intensity decreases from the central portion to the peripheral portion, the diffuser plate 17a formed of the transmissive member having the opposite transmissivity distribution characteristic in which the transmissivity increases from the central portion to the peripheral portion is arranged so as to be centered to the center portion in the emission direction of the illumination light from the LED (light source 11a), the shape of the LED (light source 11a) corresponding to the light intensity distribution characteristic is not formed as an image, so that the imaging field can be uniformly illuminated, and the site of the subject may be appropriately observed.

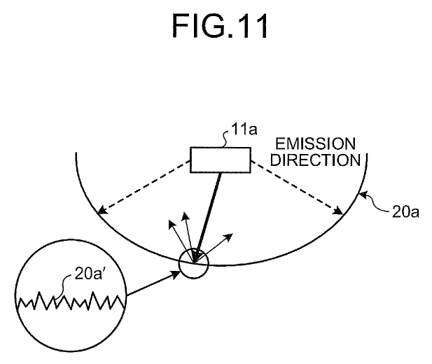

Also, although the diffuser plate 17a formed of the transmissive member through which the illumination light from the LED (light source 11a) passes is used in the fourth embodiment, the diffuser plate formed of the reflective member reflecting the illumination light may be used. FIG. 11 is a schematic configuration diagram showing a modification using a diffuser plate 20a formed of the reflective member. In this case, it is configured that the emission direction of the illumination light of the LED (light source 11a) as the light source is set to be opposite to that in the case in FIG. 2 or the like (pointing downward), and the diffuser plate 20a formed of the reflective member formed in an concave shape with a predetermined curvature is arranged in the emission direction of the illumination light from the LED (light source 11a) to illuminate the imaging field of the imaging unit 4 by reflecting the illumination light upward in a diffusing manner by the diffusing plate 20a. Here, the diffuser plate 20a is configured to reflect the illumination light from the LED (light source 11a) while diffusing the same, by a reflective inner surface thereof formed as a rough surface 20a′ as shown in an enlarged view in FIG. 11. In this case, it is desirable that the diffuser plate 20a formed of the reflective member has the reflectivity distribution characteristic in which the reflectivity increases from the central portion to the peripheral portion.

In this manner, by using the diffuser plate 20a of which reflective surface having the predetermined curvature is formed as the rough surface 20a′ also, it is possible to diffuse the illumination light from the LED (light source 11a) to uniformly illuminate the imaging field as the light having the intensity distribution substantially the same, and the object in the gas can be observed across the boundary surface without being adversely affected by the reflection on the boundary surface.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A capsule endoscope, comprising:
a capsule body to be introduced into a subject;
a center-of-gravity setting unit for setting a center of gravity so as to maintain a posture of the capsule body relative to a direction of gravity constant; and
an imaging unit arranged in the capsule body such that an imaging field having an optical axis in an oblique direction pointing upward from a horizontal direction is formed with a posture of the capsule body maintained constant relative to the direction of gravity, for imaging a body cavity image.

2. The capsule endoscope according to claim 1, wherein the center-of-gravity setting unit is formed of one member or a combination of a plurality of members built in the capsule body.

3. The capsule endoscope according to claim 1, wherein the imaging unit is arranged in the capsule body such that an imaging field is formed in a lumen direction when the capsule body is introduced into a lumen organ of the subject.

4. The capsule endoscope according to claim 3, wherein the imaging unit has a diagonal imaging optical system arranged in the capsule body with an optical axis inclined.

5. The capsule endoscope according to claim 4, wherein the diagonal imaging optical system is set so as to satisfy a condition, α<2ω/2 where a diagonal angle of the optical axis is α and a maximum angle of view is 2ω.

6. The capsule endoscope according to claim 3, wherein the imaging unit has a reflective member for inclining a direction of imaging field forward of an imaging direction of an imaging optical system in which the optical axis is set along the capsule body.

7. The capsule endoscope according to claim 6, wherein the imaging optical system is set so as to satisfy a condition, α<2ω/2 where an inclination angle in a direction of imaging field by the reflective member is α and a maximum angle of view is 2ω.

8. The capsule endoscope according to claim 1, wherein the imaging unit has imaging fields on both ends in a longitudinal direction of the capsule body.

9. A capsule endoscope, comprising:
a capsule body having a substantially cylindrical shape to be introduced into a subject;
a center-of-gravity setting unit for setting a center of gravity such that the capsule body maintains a posture relative to a direction of gravity constant without rotating in a circumferential direction, on a position eccentric from a longitudinal axis of the capsule body; and
an imaging unit arranged in the capsule body such that an imaging field having an optical axis in an oblique direction pointing upward from a horizontal direction is formed with a posture of the capsule body maintained constant relative to the direction of gravity, for imaging a body cavity image.

10. The capsule endoscope according to claim 9, wherein the center-of-gravity setting unit is formed of one member or a combination of a plurality of members built in the capsule body.

11. The capsule endoscope according to claim 9, wherein the imaging unit is arranged in the capsule body such that an imaging field is formed in a lumen direction when the capsule body is introduced into a lumen organ of the subject.

12. The capsule endoscope according to claim 11, wherein the imaging unit has a diagonal imaging optical system arranged in the capsule body with an optical axis inclined.

13. The capsule endoscope according to claim 12, wherein the diagonal imaging optical system is set so as to satisfy a condition, α<2ω/2 where a diagonal angle of the optical axis is α and a maximum angle of view is 2ω.

14. The capsule endoscope according to claim 11, wherein the imaging unit has a reflective member for inclining a direction of imaging field forward of an imaging direction of an imaging optical system in which the optical axis is set along the capsule body.

15. The capsule endoscope according to claim 14, wherein the imaging optical system is set so as to satisfy a condition, α<2ω/2 where an inclination angle in a direction of imaging field by the reflective member is α and a maximum angle of view is 2ω.

16. The capsule endoscope according to claim 9, wherein the imaging unit has imaging fields on both ends in a longitudinal direction of the capsule body.

17. A capsule endoscope, comprising:
a capsule body of which specific gravity including a built-in component is set so as to sink in liquid introduced into a subject;
a center-of-gravity setting unit for setting a center of gravity so as to maintain a posture of the capsule body relative to a direction of gravity constant in a state in which the capsule body sinks in the liquid introduced into the subject; and
an imaging unit arranged in the capsule body such that an imaging field having an optical axis in an oblique direction pointing upward from a horizontal direction is formed with a posture of the capsule body maintained constant relative to the direction of gravity, for imaging a body cavity image.

18. The capsule endoscope according to claim 17, wherein the specific gravity of the capsule body including the built-in component is equal to or larger than 1.

19. The capsule endoscope according to claim 17, wherein the center-of-gravity setting unit is formed of one member or a combination of a plurality of members built in the capsule body.

20. The capsule endoscope according to claim 17, wherein the imaging unit is arranged in the capsule body such that an imaging field is formed in a lumen direction when the capsule body is introduced into a lumen organ of the subject.

21. The capsule endoscope according to claim 20, wherein the imaging unit has a diagonal imaging optical system arranged in the capsule body with an optical axis inclined.

22. The capsule endoscope according to claim 21, wherein the diagonal imaging optical system is set so as to satisfy a condition, α<2ω/2 where a diagonal angle of the optical axis is α and a maximum angle of view is 2ω.

23. The capsule endoscope according to claim 20, wherein the imaging unit has a reflective member for inclining a direction of imaging field forward of an imaging direction of an imaging optical system in which the optical axis is set along the capsule body.

24. The capsule endoscope according to claim 23, wherein the imaging optical system is set so as to satisfy a condition, α<2ω/2 where an inclination angle in a direction of imaging field by the reflective member is α and a maximum angle of view is 2ω.

25. The capsule endoscope according to claim 17, wherein the imaging unit has imaging fields on both ends in a longitudinal direction of the capsule body.
